(19) United States
(12) Patent Application Publication
(10) Pub. No.: US 2015/0265343 A1
(43) Pub. Date: Sep. 24, 2015

(54) ANATOMICAL STRUCTURE ACCESS AND PENETRATION

(71) Applicant: RetroVascular, Inc., Pleasanton, CA (US)

(72) Inventors: Wayne Ogata, San Ramon, CA (US); Osamu Katoh, Nagoya-shi (JP)

(21) Appl. No.: 14/730,495

(22) Filed: Jun. 4, 2015

Related U.S. Application Data
(62) Division of application No. 13/042,411, filed on Mar. 7, 2011, now abandoned.
(60) Provisional application No. 61/311,204, filed on Mar. 5, 2010.

(51) Int. Cl. A61B 18/14

(52) U.S. Cl. CPC A61B 18/1492; A61B 2018/00357

(57) ABSTRACT

Methods, devices, and systems for accessing and penetrating an anatomical structure using suction force and energy ablation or mechanical penetration. In one aspect, a catheter is stabilized on the occlusion, a longitudinal member is advanced to penetrate the occlusion and recanalize the body vessel. In another aspect, a catheter is stabilized on the pericardium, ablation energy is delivered through a longitudinal member to create an opening on the pericardium.

This application is a divisional, of prior U.S. patent application Ser. No. 13/042,411, filed Mar. 7, 2011, which claims the priority benefit of U.S. Provisional Patent Application Ser. No. 61/311,204 filed on Mar. 5, 2010, which is herein incorporated by reference.

### FIELD

Embodiments of the present invention relate generally to methods, devices, and systems for anatomical structure access and penetration including but not limited to penetration of an occlusion or the pericardium.

### BACKGROUND

A chronic total occlusion (CTO) is the complete blockage of a vessel and usually has serious consequences if not treated in a timely fashion. The cause of blockage could be the deposition of atheromatous plaque, old thrombus or similar other deposits.

One of the common procedures for treating CTOs of the coronary arteries I percutaneous trans-luminal coronary angioplasty (PTCA) via a percutaneous approach. During a PTCA procedure, a small incision is typically made in the groin. A guiding catheter over a guidewire is introduced into the femoral artery and advanced to the occlusion. Frequently, with gentle maneuvering, the guidewire is able to cross the stenosis. Then, a balloon-tipped angioplasty catheter is advanced over the guidewire to the stenosis. The balloon is inflated, separating or fracturing the atheroma. Commonly, a stent is subsequently placed. Other known methods to recanalize an occluded body vessel include the subintimal tracking and reentry with side branch technique, parallel wire technique, IVUS guided technique, and retrograde approach. However, none of these methods provide satisfactory results for the most challenging of the CTOs, nor do they provide for safety of the body vessel walls from injuries caused by slipping away or deviation of the guidewire.

U.S. Pub. No. 2007/0208368A1 by Katoh et al. discloses a technique to treat CTOs by using a combined antegrade and retrograde approach. In this technique, one of the guidewires is advanced through the occlusion in an antegrade fashion while another guidewire is advanced in retrograde manner. The two guidewires are configured to engage with each other to recanalize the body vessel. However, even when using this technique, slipping or deviation of the guidewires into the subintimal space or into the inner walls of the body vessel can occur. Hence, a more effective treatment of coronary chronic total occlusions with increased safety remains a challenge.

A surgical approach towards treating CTOs is coronary bypass surgery. During a bypass procedure, the chest is opened via a sternotomy and an open field is exposed for direct access and visualization of the blood vessels to be treated. The surgical approach is quite invasive requiring general anesthesia and significant time to heal.

Additionally, when treating various ailments of the heart, access is commonly achieved either percutaneously through an incision in the groin or surgically via a sternotomy. More recently, a port access approach has been utilized whereby the heart is approached through ports or holes created through skin incisions in the chest and epicardial access to the blood vessels is achieved by puncturing the pericardium. This approach is similarly traumatic to the patient.

A number of methods of puncturing the pericardium have been utilized in the past for other applications including directly puncturing the pericardium with the use of a needle, gripping the pericardium with forceps and cutting it with a scalpel (U.S. Pat. No. 5,071,428), utilizing a device which can vacuum a “bleb” or local region of the pericardium into the device for subsequent puncturing with a needle (U.S. Pat. No. 5,827,216). One of the limitations in these approaches is the inability to accurately and safely create the puncture without potentially damaging other aspects of the heart.

What is needed are improved methods and devices for accessing the heart and penetrating tissue, as well as improved methods and devices for recanalizing an occluded vessel.

### SUMMARY

The present embodiments provide methods and devices for accessing and penetrating an anatomical structure by stabilizing an access catheter on or near the anatomical structure and subsequently advancing a longitudinal member to penetrate the anatomical structure.

According to one aspect of the present embodiments, wherein the anatomical structure is an occlusion in a vessel, a catheter is inserted into or near an occluded body vessel, the catheter comprising a proximal end, a distal end, and a suction lumen therebetween. The catheter is further configured for advancement of a guidewire therethrough. Thereafter, the distal end of the catheter is stabilized on the occlusion by applying suction or negative pressure through the suction lumen. Finally, a longitudinal member is advanced through the stabilized catheter to penetrate the occlusion and recanalize the body vessel.

In one aspect, the longitudinal member is a guidewire. In another aspect, the longitudinal member is configured to deliver energy, such as radio-frequency energy to the occlusion.

A catheter or catheter system for recanalizing an occluded body vessel is also disclosed. The system comprises a catheter in combination with a guidewire for insertion into a body vessel. The catheter comprises a proximal end, a distal end, and a suction lumen there between. The catheter is configured for advancement of a longitudinal member there through. The suction lumen is used for stabilizing the distal end of the catheter that is in contact with the occlusion, and the longitudinal member is advanced through the stabilized catheter and through the occlusion to recanalize the body vessel.

In one aspect, the catheter used in the recanalization process could be a single lumen catheter. In another embodiment, the catheter could be a multi-lumen catheter. In a multi-lumen configuration, the lumens may be arranged in a coaxial manner, or in a non-coaxial manner, such as side-by-side.

In another aspect, the catheter lumens could be defined by surfaces that are made of sufficiently strong polymer material to withstand suction pressures. Such surfaces may have a coiled structure, a braided structure, or other reinforced structure.

In another aspect, the catheter may include a balloon at its distal end to aid in targeted entry of the guidewire into the occlusion. Furthermore, the catheter may include modified tips to enhance stability of the catheter.

In yet another aspect, embodiments of the present invention may be used to recanalize an occlusion by approaching the occlusion in a retrograde or in an antegrade direction.

According to another aspect of the present embodiments, wherein the anatomical structure is the pericardium, a catheter is advanced up to a portion of the pericardium, the distal end of the catheter is stabilized on the portion of the pericardium, a longitudinal member is advanced from the catheter, wherein the longitudinal member comprises at least one energy delivery portion, energy is then delivered through the energy delivery portion of the longitudinal member to the portion of the pericardium, wherein an opening is created in the portion of the pericardium.

In one aspect, suction is delivered to the pericardium to stabilize the catheter. In another aspect, a portion of the pericardium is separated from the heart.

In yet another aspect, the energy delivery portion of the longitudinal member is configured to delivery radio-frequency energy to a portion of the pericardium.

In another aspect, the opening on the pericardium is sealed, wherein the sealing comprises cauterizing the opening by delivering energy through the energy delivery portion of the longitudinal member.

Other embodiments and variations are presented in the detailed description, as follows.

### BRIEF DESCRIPTION OF THE DRAWINGS

The invention has other advantages and features which will be more readily apparent from the following detailed description of the invention and the appended claims, when taken in conjunction with the accompanying drawings, in which:

FIG. 1 shows one embodiment of the device for anatomical structure access and penetration.

FIGS. 2A-2E show exemplary cross-sectional views of an access catheter comprising one or more lumens.

FIGS. 3A-3B show exemplary embodiments of an access catheter with various configurations of the distal portion.

FIG. 4 shows an exemplary embodiment of a longitudinal member configured to delivery energy.

FIG. 5A shows a portion of a body vessel with an occlusion.

FIG. 5B shows a guidewire being deflected by a distal cap of an occlusion into a subintimal space.

FIG. 6 shows a flow diagram of exemplary steps involved in performing the recanalization of the body vessel.

FIG. 7 is a flow diagram illustrating an exemplary method for recanalizing an occluded body vessel in an antegrade direction.

FIGS. 8A-8F show various stages of recanalization of the body vessel in an antegrade direction.

FIG. 9 is a flow diagram illustrating an exemplary method for recanalizing an occluded body vessel in a retrograde direction.

FIGS. 10A-10G show various stages of recanalization of the body vessel in a retrograde direction.

FIG. 11 shows an exemplary embodiment of a device for pericardium access and penetration.

FIG. 12 is a flow diagram illustrating an exemplary method for accessing and penetrating the pericardium.

FIGS. 13A-13C show various stages of pericardium access and penetration.

FIG. 14 shows an exemplary embodiment of a device for pericardium access and penetration utilizing two longitudinal members in a bipolar energy delivery configuration.

### DETAILED DESCRIPTION

Although the detailed description contains many specifics, these should not be construed as limiting the scope of the invention but merely as illustrating different examples and aspects of the invention. It should be appreciated that the scope of the invention includes other embodiments not discussed herein. Various other modifications, changes and variations which will be apparent to those skilled in the art may be made in the arrangement, operation and details of the method, device, and system of the present invention disclosed herein without departing from the spirit and scope of the invention as described here.

Embodiments of the present invention relate generally to methods, devices, and systems for penetrating an anatomical structure, and in particular to using suction or negative pressure to stabilize a catheter tip on a surface of the anatomical structure in order to penetrate said anatomical structure using various means such as energy ablation and/or mechanical penetration.

As referred to herein, the term anatomical structure is meant to include any organ, or part of organ having a wall with an outer surface. The wall may comprise a capsule, muscularis, a membrane, endothelial layers of a hollow organ or vessel, or other conglomerates of tissue cells. Additionally, the anatomical structure is meant to include any organic or inorganic deposits within a body region such as deposition of atheromatous plaque, old thrombus, or other deposits.

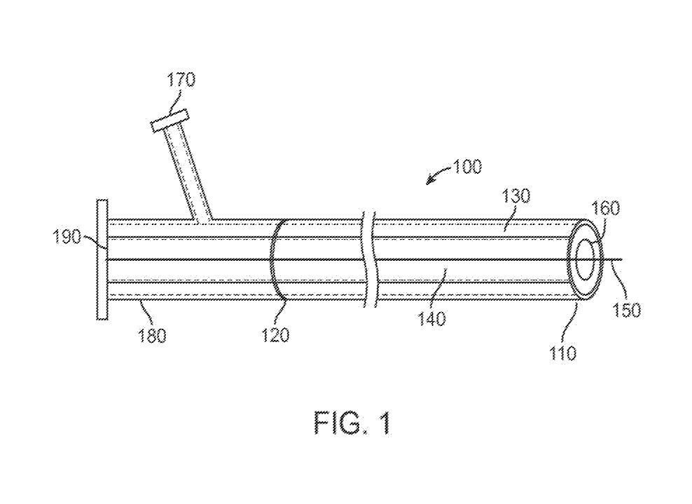

One embodiment of a device for anatomical structure access and penetration is shown in FIG. 1, wherein an access catheter 100 comprises a distal end 110, a proximal end 120, and a first lumen 130 there between. The first lumen 130 may be configured to deliver suction or negative pressure to stabilize the catheter 100 within an anatomical region and/or on an anatomical structure. The embodiment shown in FIG. 1 may further comprise a second lumen 140 disposed within the first lumen 130. The second lumen 140 may be configured to advance a longitudinal member 150 through the catheter 100. The second lumen 140 comprises an opening 160 at the distal end 110 of the catheter 100 for further passage of the longitudinal member 150 into an anatomical region. In alternative embodiments, the second lumen may be disposed outside of the first lumen, or the first lumen itself may also serve as a second lumen, as will be described in further detail below.

The catheter 100 is configured to attach to a hub 180. The hub 180 may comprise a suction port 170 configured to communicate suction to the first lumen 130. Using the suction port 170, suction force may be applied to a portion of the anatomical structure to stabilize the catheter 100. The hub 180 may further comprise an insertion port 190 configured to introduce the longitudinal member 150 into the second lumen 140 of catheter 100. The hub 180 may be detachable from the catheter 100 or it may be incorporated as a part of the catheter 100.

In one embodiment, the suction port 170 may be connected to a locking syringe (not shown) which generates negative pressure to create the suction applied to the portion of the anatomical structure through the first lumen 130. In another embodiment, the suction port 170 may be connected to a mechanized vacuum pump system (not shown) acting through an isolation valve, wherein the mechanized vacuum pump system generates the suction force. It is further contemplated that suction may be achieved by using various other means known in the art to create a pressure differential between the first lumen 130 and the anatomical structure.

It is noted that the first lumen may be subject to a large force, as applied at the suction port 170. In one embodiment, to ensure that the first lumen has sufficient strength to prevent collapse in response to the large applied suction force or back-up force, the walls surrounding the lumen may be made of any suitable material such as various high strength polymers, including polyimide and polyester. Additionally or alternatively, the walls may comprise reinforcing fibers or wires arranged structurally in coiled or braided configurations. Additionally, the exterior of the catheter may be coated or configured as a helical surface or a smooth surface to aid in advancement of the catheter through an anatomical structure.

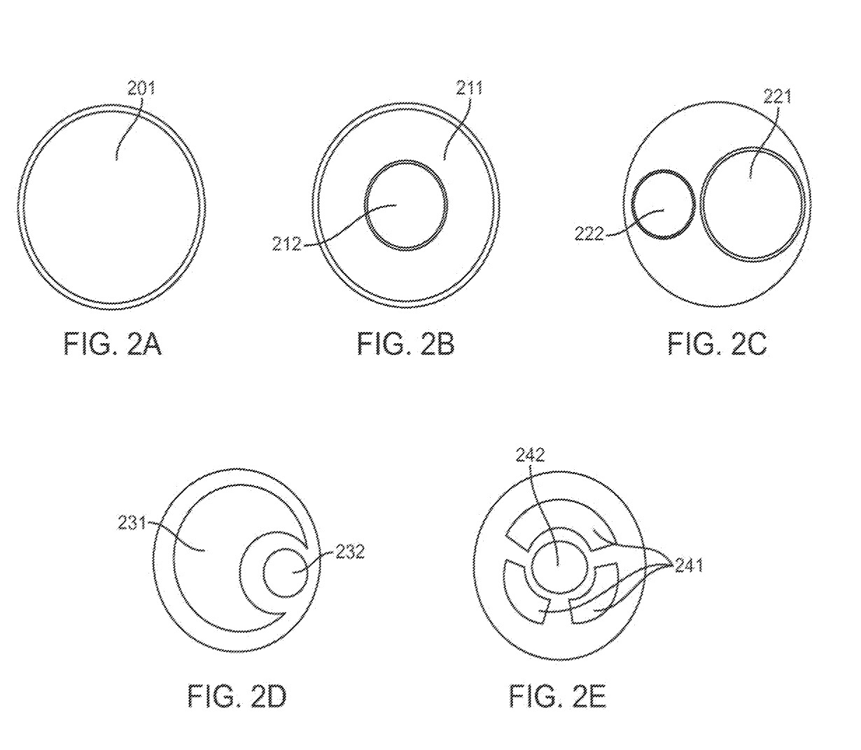

As described above, the first lumen and the second lumen may be combined or may be separate, and several examples are shown in cross-sectional view in FIGS. 2A-2E. In the exemplary embodiment of FIG. 2A, the first lumen and the second lumen are configured as a single combined lumen 201. In the exemplary embodiment of FIG. 2B, the first lumen 211 and the second lumen 212 are configured as separate but substantially coaxial lumens, as shown in the cross-sectional view. In the exemplary embodiment of FIG. 2C, the first lumen 221 and the second lumen 222 are configured as substantially separate side-by-side lumens. In the exemplary embodiment of FIG. 2D, the first lumen 231 and the second lumen 232 are substantially separate, and they are not concentric, wherein the second lumen 232 is disposed substantially off-axis from the catheter axis. In yet another embodiment, the catheter may comprise more than two lumens. FIG. 2E shows a cross-sectional view of one embodiment of a four lumen configuration, with three lumens 241 configured to deliver suction force shown disposed around a lumen 242 configured to advance a longitudinal member. It is noted that these embodiments are exemplary, and that the catheter may comprise one or more lumens configured to deliver suction force and/or lumens to accommodate or advance the longitudinal member or other elements of various configurations.

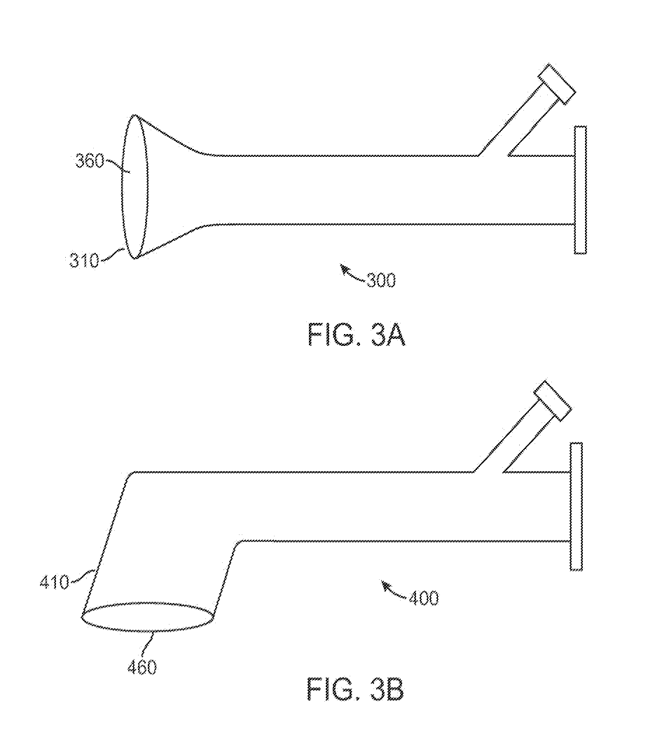

The distal end of the catheter may assume various configurations to facilitate access and interaction with the targeted anatomical structure. Referring now to FIG. 3A and FIG. 3B, where two embodiments of the catheter are shown. As seen in FIG. 3A, the distal end 310 of the access catheter 300 may be configured to assume a tapered configuration, wherein the diameter of the distal opening 360 is larger than the diameter of the catheter shaft. Having a larger distal opening may be advantageous since it allows the access catheter 300 to transfer suction force or back-up force to a greater area of the anatomical structure and therefore it may improve the stabilization of the catheter on the anatomical structure. Another embodiment of the catheter is shown in FIG. 3B, where the distal end 410 of the access catheter 400 is configured to be at an angle relative to the axis of the body of the catheter 400. The angled configuration may be advantageous such that it may facilitate access to targeted anatomical structure. For example, the access catheter 400 with an angled distal end 410 may be inserted substantially parallelly to the anatomical structure, wherein the angled distal end 410 enables the distal opening 460 to interact with the targeted anatomical structure while the axis of the body of the access catheter 400 remains substantially parallel to a portion of the anatomical structure.

The angle of the distal end 410 may be configured to various degrees depending on the operation and/or the anatomy of the operating body region. In one embodiment, the angle of the distal end 410 may be configured as up to 45 degree with respect to the axis of the body of the access catheter 400. In another embodiment, the angle may be configured as up to 90 degree with respect to the axis of the body of the access catheter 400. In yet another embodiment, the angle may be configured as up to 180 degree with respect to the body of the catheter 400.

The angle of the distal end 410 may be pre-configured by using a shape memory material or other means known in the art during manufacture, or it may be configured by the operator prior to, or during the operation. It is further contemplated that the angle of the distal end 410 may be variable and/or adjusted by the operator.

The access catheter may be configured with elastic properties such that at least a portion of the access catheter is mutable. In one embodiment, the access catheter may be constructed of flexible material, such that the access catheter may pass through tortuous body regions. Furthermore, the mutable configuration of the access catheter may allow it to dynamically assume various curvatures and to regain its original shape depending on the type of support positioned within.

Additionally, the access catheter may comprise a modified distal end to enhance stability and/or aid in the advancement of the access catheter. Examples include a distal end comprising a metallic tip, a sharpened or tapered tip, a serrated tip, a screw tip, a soft tip, or a helical tip which can be rotated to advance into an anatomical region.

Additionally and optionally, it is envisioned that the access catheter may comprise additional elements such as an anchoring mechanism that is configured to attach to a portion of the anatomical structure. The anchoring mechanisms such as barbs, clips, or locking mechanisms may be disposed on or near the distal end of the access catheter. The anchoring mechanisms may attach the access catheter to a portion of the anatomical structure to further stabilize the access catheter onto the anatomical structure.

The longitudinal member 150 may be various devices configured to penetrate the anatomical structure. It one embodiment, the longitudinal member may be a guidewire, catheter, micro-catheter, or a dilating catheter, a tubular element, a needle, or they may assume any configuration capable of penetrating through the target anatomical structure to create an opening. Additionally, it is contemplated that a cross-sectional area of the longitudinal member may be configured to progressively increase from the distal end towards the proximal end. The tapered configuration may be advantageous in that the narrow distal end may be configured to facilitate penetration, whereas the larger tail is configured to allow a physician to manipulate the longitudinal member during the operation. Alternatively and optionally, a cross-sectional area of the longitudinal member may be configured to be substantially unchanged throughout the lengths of the longitudinal member.

It is noted that the flexibility of the longitudinal member may vary over its length. In one embodiment, the distal end of the longitudinal member is substantially flexible, and the flexibility progressively decreases towards the tail.

The longitudinal member may comprise core wires of different types and configurations for providing improved torque and easy maneuvering during penetration. In one embodiment, such a core wire may be configured to have a cross-section with an aspect ratio of approximately one. In another embodiment, the core wire may be configured to have a cross-section with an aspect ratio of less than one. In one embodiment, the core wire is configured to have a substantially flat cross-section. It is contemplated that the core wires may be stainless steel, Nitinol, Elgiloy, platinum, iridium, tantalum, titanium, cobalt, chromium, tungsten, combinations thereof, or other biologically compatible materials.

The distal end of the longitudinal member may assume any configurations that enable penetration and/or opening creation. In one embodiment, the distal end of the longitudinal member may be configured as a deflectable tip. In another embodiment, the distal end may be configured as a bevel tip, screw tip, a soft tip, or a helical tip.

Additionally, the longitudinal member 150 may be configured as an energy delivery element wherein an energy modality is delivered to the body region to ablate the targeted anatomical structure such that a portion of the anatomical structure is substantially destroyed or weakened and an opening may be created.

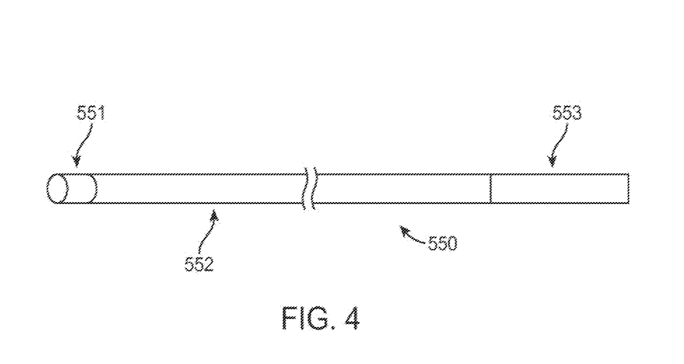

As seen in FIG. 4, a longitudinal member 550 configured to deliver energy to ablate an anatomical structure comprises an energy delivery portion 551, an energy connector 553, and an insulated portion 552 disposed there between. In one embodiment where an energy modality used to ablate a portion of an anatomical structure is radio-frequency (RF) energy, one or more RF electrodes are placed on the energy delivery portion 551 of the longitudinal member 550 and the insulated portion 552 may be configured to provide protection to the adjacent areas. The insulated portion 552 may be constructed of Teflon or ceramic. Alternatively, an insulated cap or disc may be added to the longitudinal member 550 to provide insulation.

The energy connector 553 may be connected to an appropriate RF generator (not shown) to provide RF energy using one or more connectors. The generator may be designed to monitor and measure any number of parameters including but not limited to time, temperature, energy, and impedance.

The use of RF energy can be achieved by either a monopolar arrangement or a bipolar arrangement. In a typical monopolar arrangement, one or more electrodes may be disposed on the longitudinal member and a large plate may be placed outside the body. Alternatively, two or more electrodes may be used to deliver the RF energy, such that controlled energy deployment is achieved using a bipolar arrangement of the electrodes.

The electrodes in the bipolar arrangement may be referred to as the anode and cathode, or as the return and active electrodes. The electrodes may be arranged in an array (multiple electrodes), where the electrode arrangement provides better control over the depth of penetration of the RF field and thereby provides the ability to control the tissue temperature. The anode and the cathode may be disposed on the same longitudinal member. Alternatively, one electrode may be disposed on a first longitudinal member, whereas the other electrode may be disposed on a second longitudinal member or an electrode wire. Both the first and the second longitudinal members may be disposed within the access catheter, or alternatively, the second longitudinal member may be disposed anywhere on or near the targeted anatomical structure.

It is noted that the electrodes may assume various configurations. In one embodiment, an active electrode is configured to have a smaller surface area than a return electrode. This allows the active electrode to generate a current density that is sufficiently high to cause radiofrequency sparks crossing over to the return electrode, while at the same time allowing the return electrode surface area to be sufficiently large so as to maximize its contact with the anatomical structure and attract sparks from the active electrode. Another advantage of such an embodiment is that the return electrode will likely not reach as high temperatures as the active electrode. In one embodiment, the ratio of the return electrode surface area to the active electrode surface area is configured to be in the range of about 50:1 to 2:1, and preferably about 10:1. In one embodiment, the return electrode is configured in a pigtail design to increase surface area contact with the occlusion. In another embodiment, a plurality of return electrodes may be configured to expand outwardly in order to spread out and increase surface area contact with the anatomical structure.

While the above embodiments refer to the use of RF energy for ablation, it should be noted that other energy modalities may be used as well, for example ultrasound energy. In one embodiment, the energy delivery portion of one or more longitudinal members may comprise one or more ultrasound transducers, instead of or in addition to RF electrodes. The ultrasound transducers are configured to provide ultrasound energy for ablating an anatomical structure. It is further contemplated that the energy delivery portion may be configured to deliver thermal energy, whereby the thermal energy radiating from the energy delivery portion may ease the penetration and/or the advancement of the longitudinal member. Other energy modalities may include microwave and laser.

In a generic operation of the present embodiments, the access catheter 100 is advanced into a body region such that the distal end 110 of the access catheter may be positioned on or near an anatomical structure. Thereafter, suction force may be delivered through the suction port 170 and transmitted through the first lumen 130 of the access catheter. The suction force may then be exerted on to a portion of the anatomical structure to stabilize the access catheter on to, or near the anatomical structure. Optionally, the suction force may draw or capture a portion of the anatomical structure into the access catheter 100. Additionally and optionally, the access catheter 100 may be manipulated to lift, move, or separate a portion of the anatomical structure from one or more underlying structure. Thereafter, at least one longitudinal member 150 may be advanced through the second lumen 140 of the access catheter. The longitudinal member 150 may be used to create an opening by ablating a portion of the anatomical structure through energy delivery to the anatomical structure. Alternatively or additionally, the longitudinal member may be used to mechanically puncture or otherwise create an opening. The longitudinal member or other apparatus may be advanced through the opening to carry out further operations.

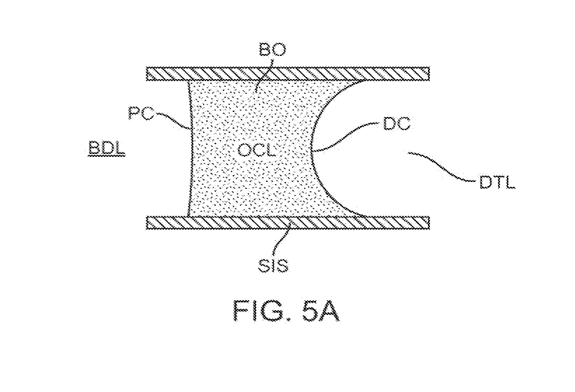

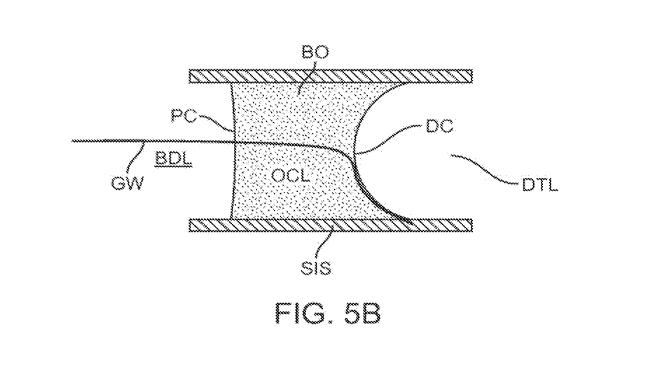

One exemplary application of embodiments of the present invention is recanalization of an occluded body vessel, in particular, the recanalization of chronic total occlusion. Referring now to FIGS. 5A-5B, where a schematic diagram of a portion of an occluded body vessel BDL is shown. The body vessel BDL could be any vessel or artery in which blood flows through the hollow tubular cavity. An occlusion OCL within the body vessel BDL may obstruct the blood flow and could have fatal consequences. Typically, treatment procedures may involve approaching the occlusion from an antegrade and/or a retrograde direction. The occlusion OCL comprises a distal cap DC, a proximal cap PC, and an occlusion body BO therebetween. In the combined, antegrade-retrograde approach, the distal cap DC is typically approached from a retrograde direction whereas the proximal cap PC is typically approached from an antegrade direction. The occlusion OCL could be atheromatous plaque, old thrombus, or similar other deposit. One method of recanalizing the occlusion OCL is by using guidewire techniques, wherein a guidewire penetrates the occlusion OCL and a catheter recanalizes the vessel.

Depending on the type and the composition of the occlusion OCL, it may be difficult to successfully penetrate the occlusion OCL using standard guidewire techniques. In particular, the distal cap DC of the occlusion may be composed of dense, fibrous tissue with fibrocalcific regions. Generally, it may be necessary to use a guidewire of sufficient rigidity to successfully penetrate the distal cap DC. Also, it may generally be necessary to apply substantial force in order to penetrate the distal cap DC of the occlusion and recanalize the body vessel.

In particular, when traversing the occlusion in an antegrade direction, it has been a challenge to successfully penetrate and traverse the distal cap DC and enter the distal true lumen DTL without entering into subintimal space SIS. This is so because, combined with its fibrous composition, the distal cap DC of the occlusion often assumes a morphology that renders penetration difficult, as the guidewire is likely to be deflected away from the fibrous interior surface of the distal cap DC (see FIG. 5A). The difficulties in penetrating the distal cap DC of the occlusion often lead to the guidewire slipping away from the interior surface of the distal cap DC and entering into subintimal space SIS (see FIG. 5B). The penetration of the subintimal space SIS by a guidewire GW may lead to the puncturing of the wall of the body vessel, which may cause bleeding as well as other undesirable side effects. Furthermore, by penetrating the subintimal space SIS instead of the distal cap DC, it is substantially more difficult for a catheter to advance into the distal true lumen DTL to complete the recanalization.

The present embodiments may be configured to reduce the likelihood of a guidewire slipping away from a portion of the occlusion, such as the distal cap DC, into the subintimal space SIS in the process of recanalization of an occluded body vessel. Specifically, the present embodiments may employ suction force to stabilize the distal end of a catheter on a portion of the occlusion, such as an interior or exterior surface of the distal cap DC of the occlusion. With the distal end of the catheter substantially stabilized on the portion of occlusion, a longitudinal member may be advanced to penetrate the occlusion. Thereafter, a catheter may be advanced through the occlusion to recanalize the body vessel.

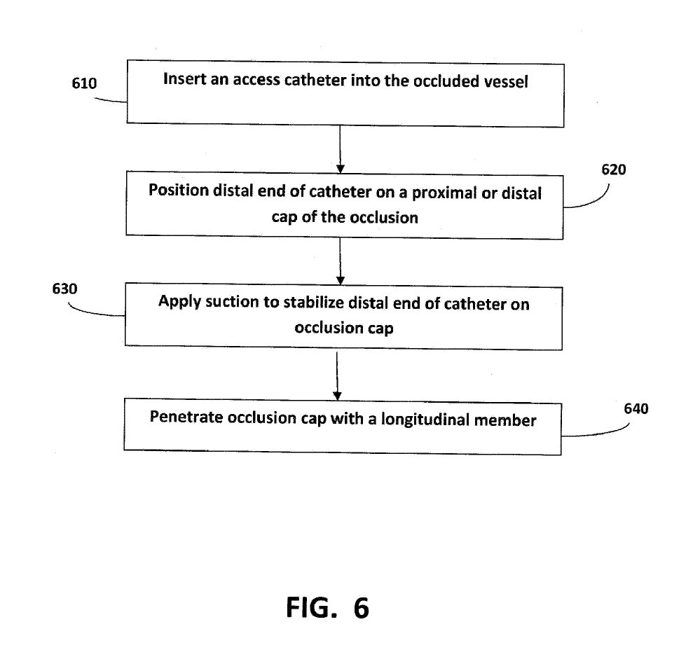

A recanalization method is shown schematically as a flow diagram in FIG. 6, according to one embodiment. At step 610, an access catheter is inserted into an occluded body vessel. At step 620, the distal end of the catheter is placed on a portion of the occlusion, such as the distal cap DC or proximal cap PC of the occlusion. At step 630, suction is applied through suction port connected to a first lumen. Due to the force exerted by suction, the catheter is stabilized on the occlusion. Thereafter, at step 640, with the distal end of the catheter stabilized on the portion of the occlusion, a longitudinal member such as a guidewire is advanced through the catheter to penetrate the portion of the occlusion. Optionally, the catheter may be advanced through the occlusion to recanalize the body vessel.

As described above, stabilization of the distal end of the catheter on the portion of the occlusion reduces the possibility of longitudinal member slipping away from the portion of the occlusion or being deflected into the subintimal space SIS. Additionally, the stabilization of the distal end of the catheter provides increased back-up force to allow the longitudinal member to more effectively penetrate the occlusion.

It is contemplated that the methods, devices, and systems disclosed herein may be used by approaching the occlusion in the antegrade direction or in the retrograde direction.

In one embodiment of an antegrade approach, the access catheter approaches the occlusion OCL by first advancing through the body vessel BDL in an antegrade direction towards the proximal cap PC of the occlusion OCL. The catheter then crosses the proximal cap PC, advances through the body BO of the occlusion, and exits the occlusion OCL through the distal cap DC of the occlusion to achieve recanalization of the vessel BDL.

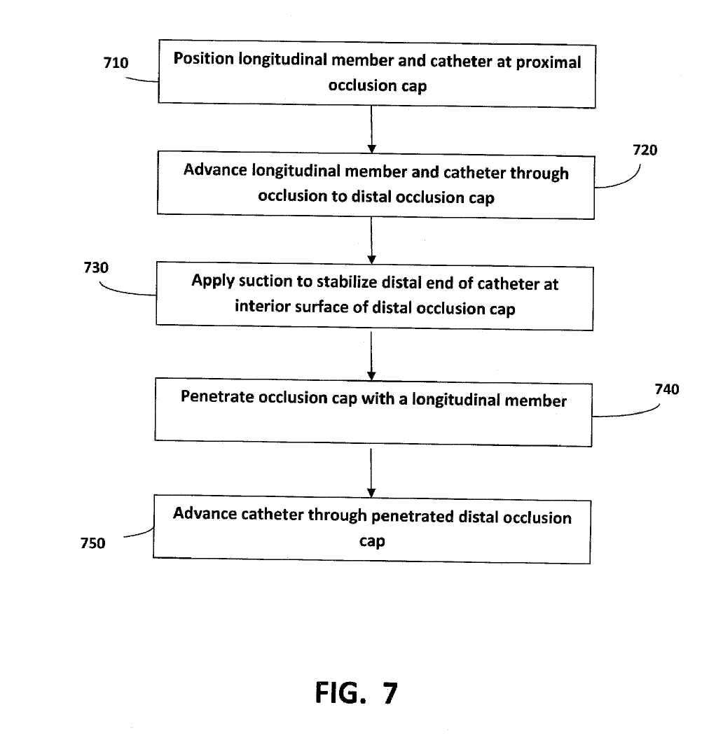

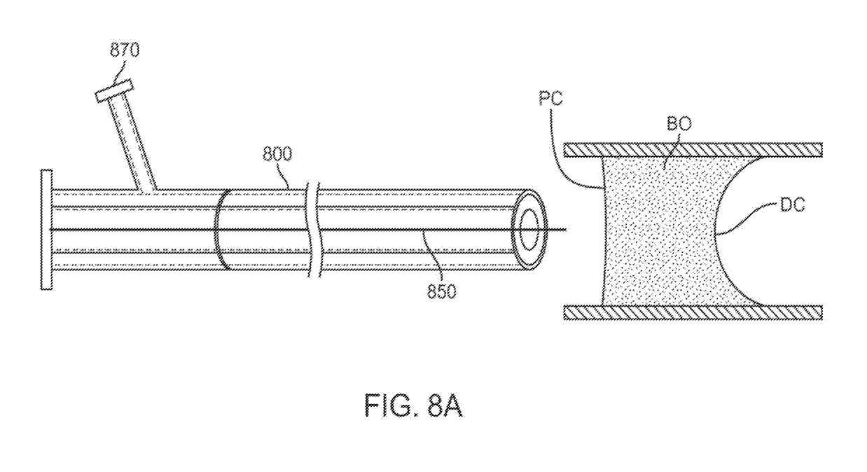

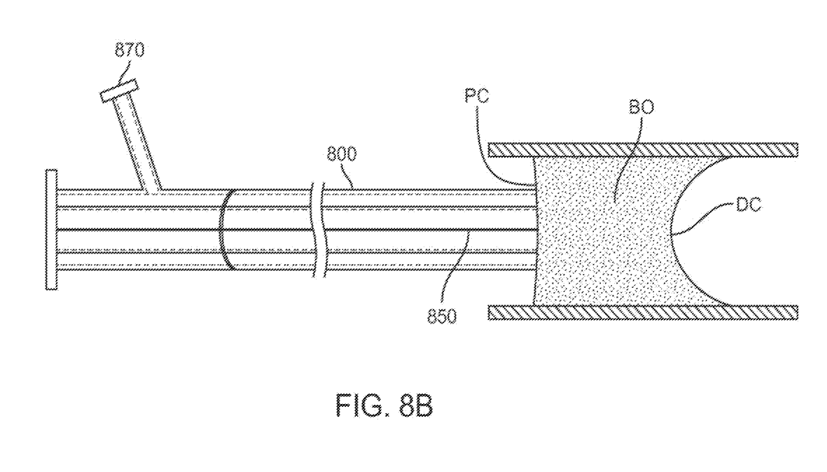

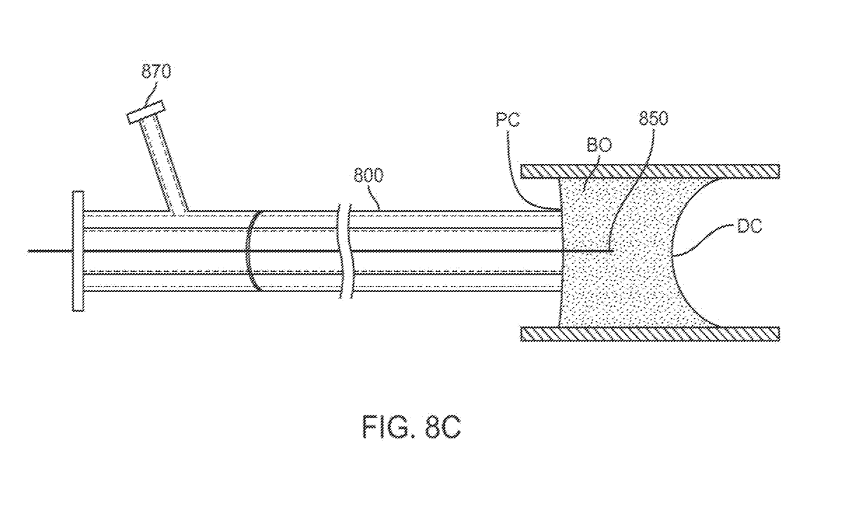

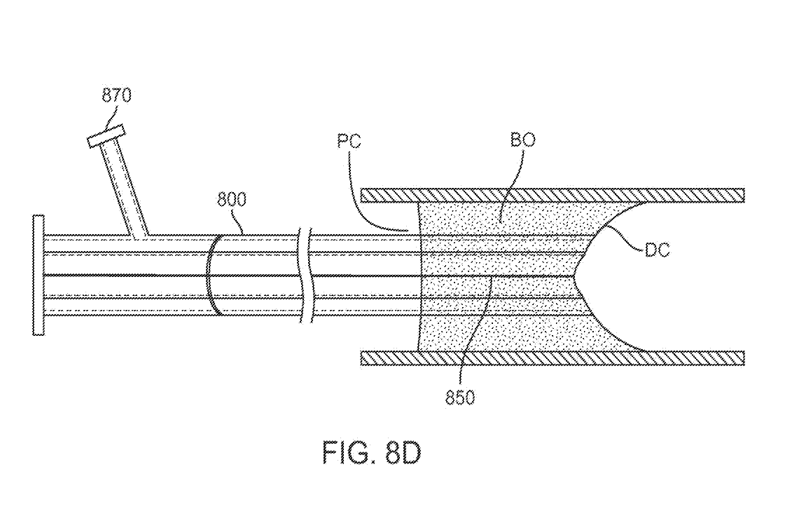

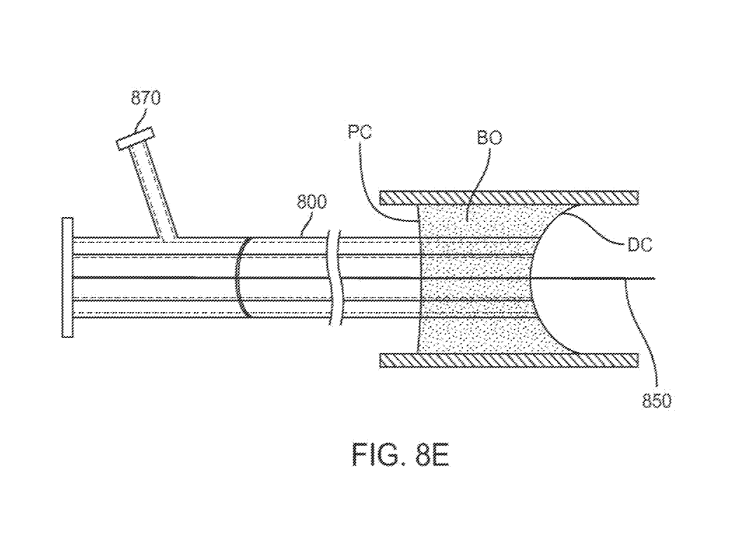

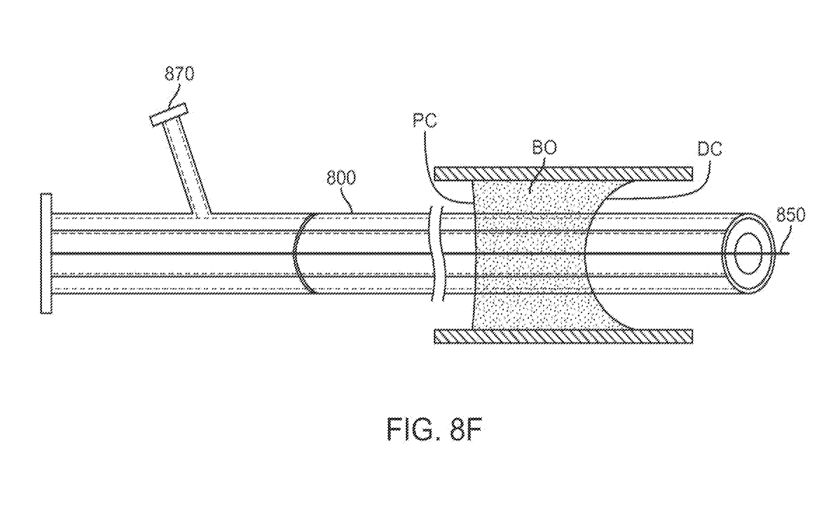

The antegrade approach is shown schematically as a flow diagram in FIG. 7, with reference to FIGS. 8A-8F. At step 710, the longitudinal member 850 such as a guidewire is inserted into the occluded body vessel BDL. Thereafter, the catheter 800 is inserted into the body vessel, tracking the longitudinal member 850 until the catheter 800 is positioned near or at the proximal cap PC of the occlusion OCL (FIGS. 8A-8B). At step 720, the longitudinal member 850 and catheter 800 advance through to penetrate the proximal cap PC of the occlusion and move through the body BO of the occlusion until the distal end 810 of the catheter is placed on an interior surface of the distal cap DC of the occlusion. At step 730, suction is applied through the suction port 870 to the interior surface of the distal cap DC of the occlusion to stabilize the distal end 810 of the catheter on the distal cap DC (FIGS. 8C-8D). At step 740, the longitudinal member 850 advances through the now stabilized catheter 800 to penetrate the distal cap DC of the occlusion (FIG. 8E) and to create an opening to facilitate recanalization. Optionally, at step 750, the catheter advances through the penetrated distal cap DC of the occlusion and thereby recanalizes the body vessel BDL (FIG. 8F). Step 750 may optionally be performed after releasing suction.

In one embodiment of a retrograde approach, the catheter approaches the occlusion OCL by first advancing through the body vessel BDL in a retrograde direction towards the distal cap DC of the occlusion OCL. The catheter then crosses the distal cap DC, advances through the body BO of the occlusion, and exits the occlusion OCL through the proximal cap PC of the occlusion to achieve recanalization of the vessel BDL.

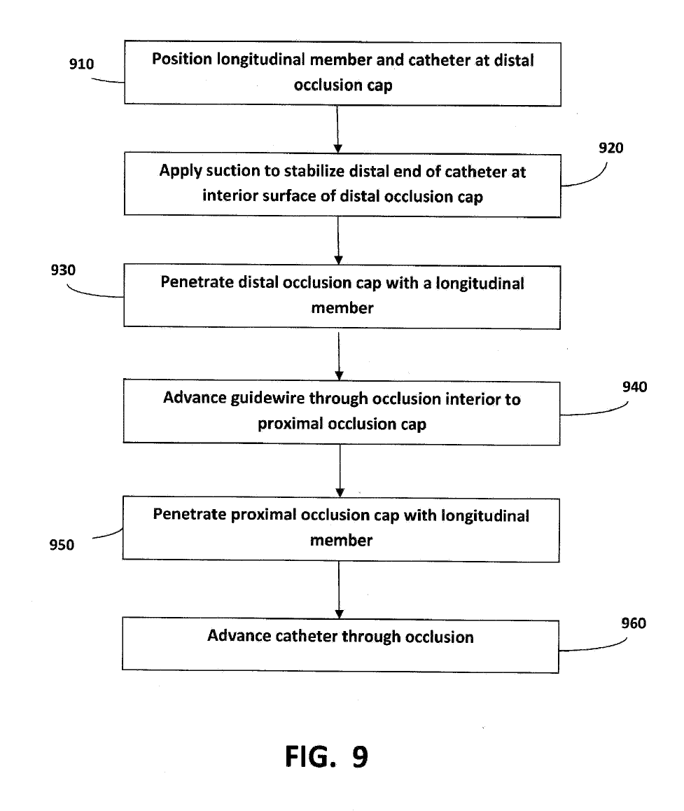

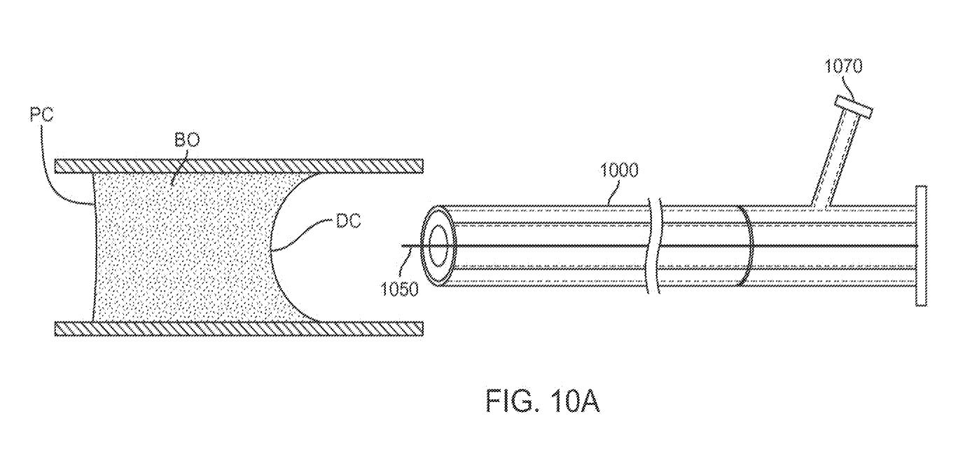

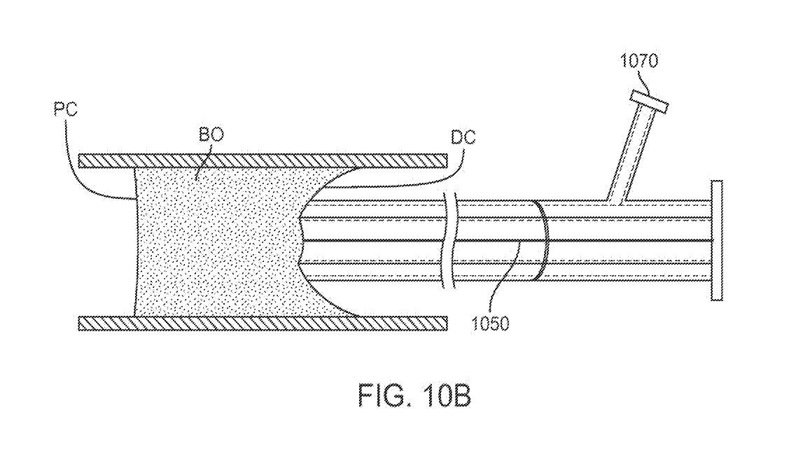

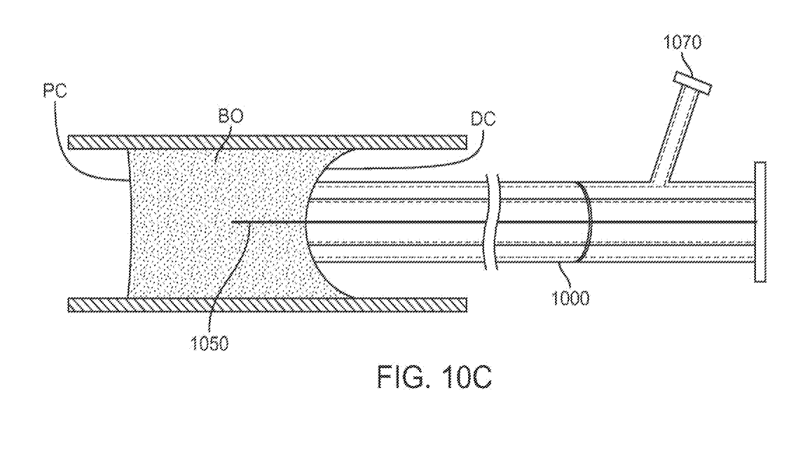

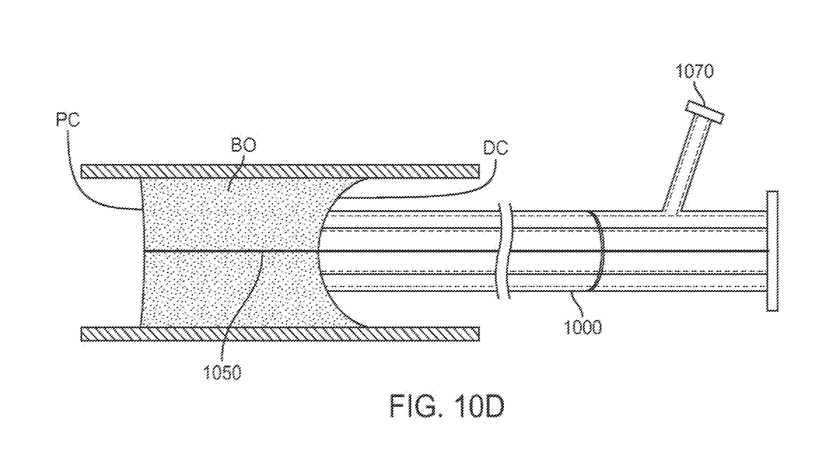

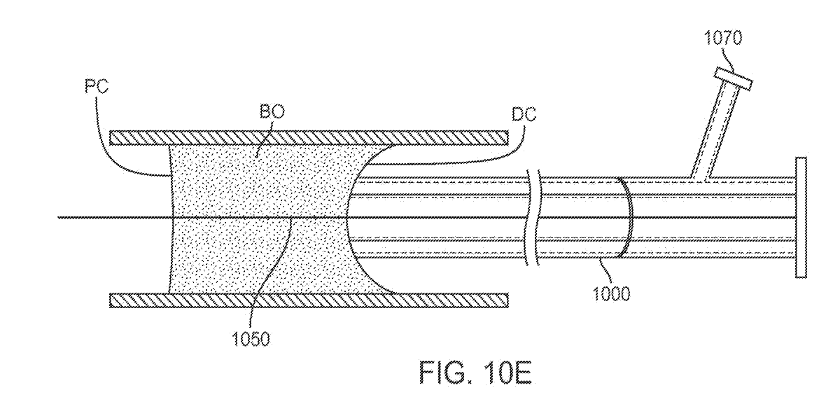

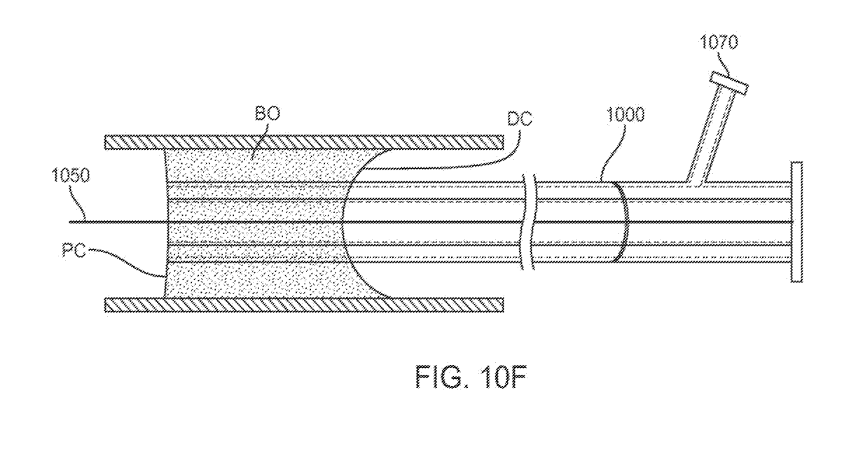

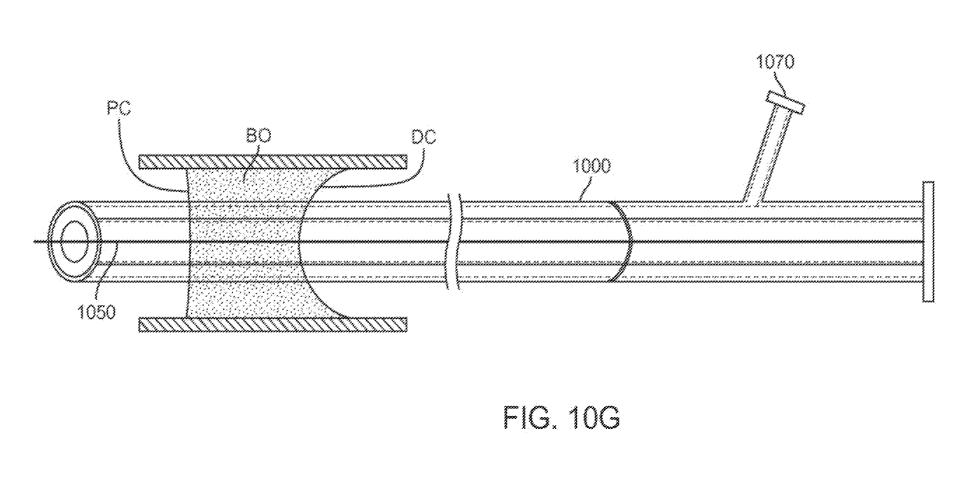

The retrograde approach is shown schematically as a flow diagram in FIG. 9, with reference to FIGS. 10A-10E. At step 910, the longitudinal member 1050 such as a guidewire and catheter 1000 are inserted and advanced until they are positioned near an exterior surface of the distal cap DC of the occlusion (FIG. 10A). At step 920, suction is applied through the suction port 1070 to the exterior surface of the distal cap DC of the occlusion to stabilize the distal end of the catheter 1000 on the exterior surface of the distal cap DC (FIG. 10B). At step 930, the longitudinal member 1050 advances through the catheter 1000 to penetrate the distal cap DC of the occlusion and to create an opening (FIG. 10C). At step 940, optionally after releasing suction, the longitudinal member 1050 advances through the penetrated distal cap DC of the occlusion and traverses through the body BO of the occlusion (FIG. 10D). At step 950, the longitudinal member 1050 penetrates the proximal cap PC of the occlusion (FIG. 10E), and at optional step 960 the catheter may advance through the occlusion and recanalize the body vessel (FIGS. 10E-10G).

Alternatively, it is contemplated that in the antegrade approach and the retrograde approach, the catheter may be placed anywhere along or outside the occlusion to provide the suction force or back-up force to stabilize the distal end of the catheter such that the longitudinal member penetrates the occlusion.

It is noted that the present embodiments may be further configured to access the distal true lumen DTL in the event the longitudinal member enters the subintimal space SIS. In such a configuration, the catheter may be advanced and repositioned against the occlusion from the subintimal space SIS. Thereafter, suction force or back-up force may be applied to stabilize the catheter and the longitudinal member may be used to penetrate an intimal layer back into the occlusion OCL. In the case where the longitudinal member is positioned beyond the occlusion, the suction force or the back-up force may be applied to stabilize the catheter and the longitudinal member, such that the longitudinal member may advance back into the distal true lumen DTL to complete the recanalization.

Although the longitudinal member configured for recanalization are exemplarily shown and described as a guidewire, it is further contemplated that the present embodiments may be combined with the use of various energy modalities delivered through one or more longitudinal members for recanalizing occluded lumens. It has been observed that using energy, for example, RF energy to ablate or alter the tissue in a controlled fashion is beneficial in crossing hard to cross lesions. The RF energy may be delivered to the occlusions in a monopolar arrangement or bipolar arrangement. In particular, the methods and systems to recanalize occlusions using a bipolar electrode arrangement are disclosed in PCT International App. Ser. No. PCT/US2008/077403 by the same inventors and incorporated herein by reference in its entirety.

Additionally and optionally, the access catheter may comprise a balloon attached to the distal end of the catheter. In this configuration, the catheter may be positioned with the distal end of the catheter on the surface of the occlusion. The balloon may then be inflated through a separate inflation lumen to align the catheter with the axis of the occluded body vessel. Thereafter, suction may be applied to stabilize the catheter on the occlusion and the longitudinal member may be advanced through the occlusion, as described above.

Another exemplary application of the embodiments of the present invention is accessing the pericardial space and/or the heart by stabilizing the access catheter on a surface of the pericardium and creating an access opening on the pericardium without puncturing the heart.

Creating an access opening on the pericardium may be required since various elements and materials may be introduced into the pericardial space and beyond for treatment and diagnosis. For example, site-specific drugs may be delivered through the access opening to the heart and coronary arteries. Furthermore, the access opening may facilitate transmysocardial revascularization, pacemaker lead implantation, defibrillator lead placement, placement of arterial bypass graphs or the like.

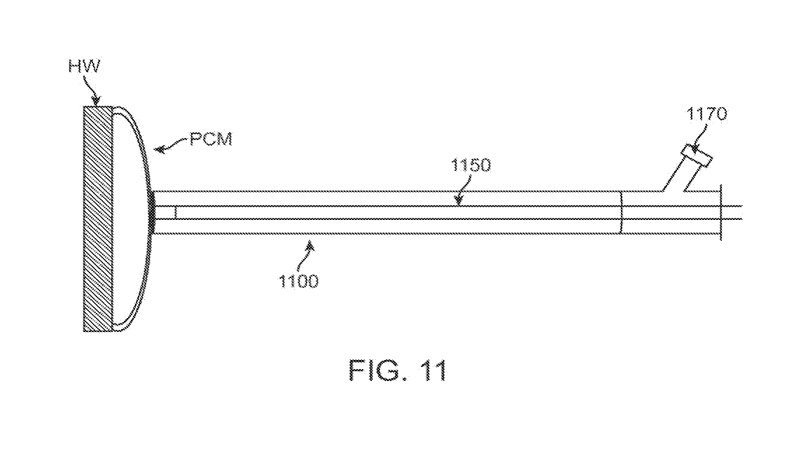

Referring now to FIG. 11, where one embodiment of a pericardial access device is shown. The device comprises an access catheter 1100 configured to approach the pericardium PCM by percutaneously positioning a distal end of an access catheter 1100 over the parietal pericardium. Positioning can be achieved either by a transthoracic or a subxiphoid approach beneath the sternum or from the abdominal cavity through the diaphragm, or the like.

The access catheter 1100 may further comprise a suction port 1170 configured to communicate suction through a first lumen of the catheter 1100, such that the catheter 1100 stabilize the catheter 1100 on to a portion of the pericardium.

The suction force as imparted onto the portion of the pericardium may allow the operator to separate the parietal pericardium from visceral pericardium by pulling, drawing, moving, lifting or otherwise manipulating the catheter 1100 with a portion of the parietal pericardium substantially attached to the catheter 1100 by suction. The separation the parietal pericardium from the visceral pericardium may enlarge the available volume of the pericardial space therebetween to increase safety and create additional working space.

The longitudinal member 1150 of an embodiment configured to access the pericardial space may comprise an energy delivery element as described above. In one embodiment, the longitudinal member 1150 may be configured to deliver RF energy to ablate or otherwise affect a portion of the pericardium to create the access opening to the pericardial space and the heart HW. Although mechanical means to create the access opening such as a guidewire is also contemplated, using RF energy to create the access opening may be advantageous to mechanical means since energy ablation may prevent accidental puncture of the heart HW. The use of RF energy has the added benefit of cauterizing the opening to aid in sealing upon completion of the procedure. Additionally, it is contemplated that energy ablation and mechanical penetration may be used in tandem.

The use of RF energy can be achieved by either a monopolar arrangement or a bipolar arrangement as described above. In a bipolar arrangement, where at least two electrodes are used (anode and cathode) it is envisioned that the second electrode may be placed at any number of locations in or around the heart. For example, an electrode wire or a second longitudinal member may be placed inside a heart chamber or blood vessel. The electrodes may also be placed on the same longitudinal member.

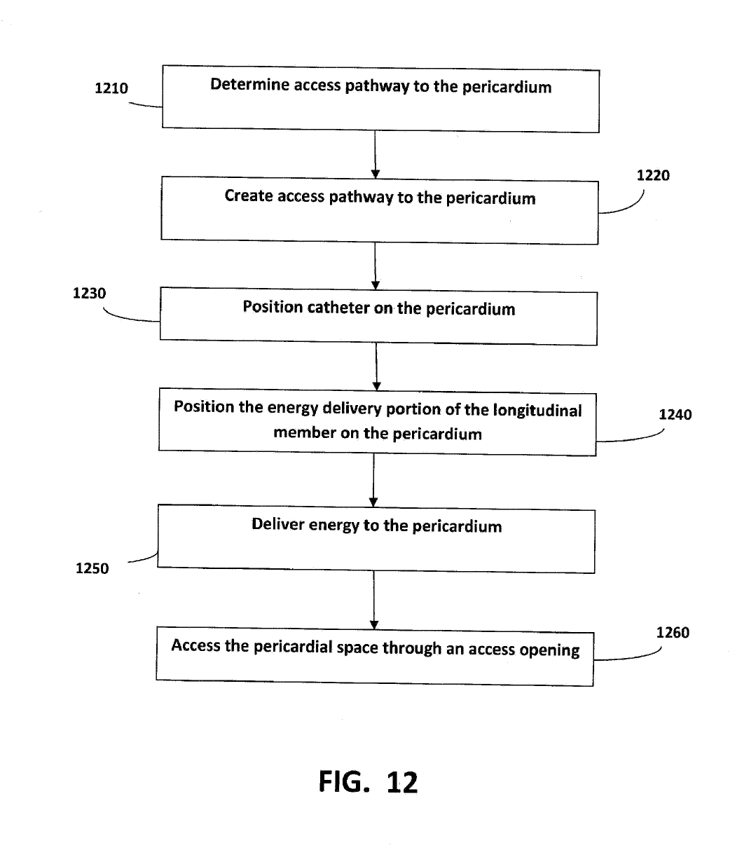

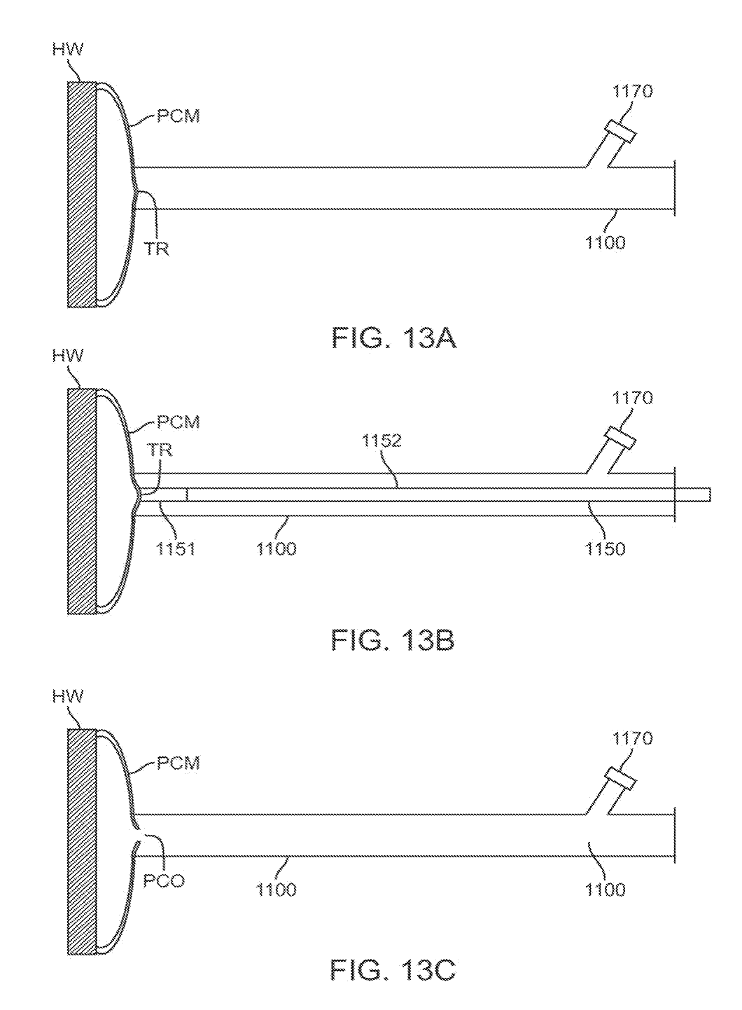

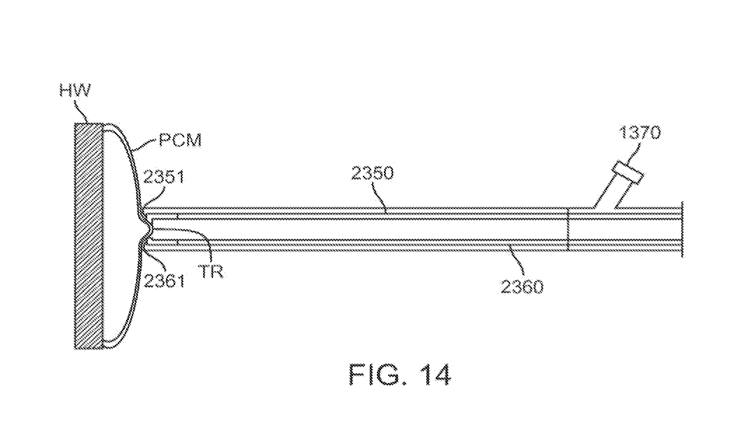

One exemplary operation of pericardial access is shown schematically as flow diagram in FIG. 12, with references to FIGS. 13A-13C. At step 1210, safe and clear access to the heart is determined via the use of CT scans or other imaging modalities. At step 1220, the access to the pericardium may be created such that the access catheter 1100 is percutaneously positioned over the pericardium PCM. At step 1230, and as seen in FIG. 13A, the access catheter 1100 may be positioned on or near the pericardium PCM and suction force may be applied through the suction port 1170. In one embodiment, as seen in FIG. 13A, a targeted portion TR of the pericardium may be subjected to the suction force and may be isolated or captured into the access catheter 1100. Additionally and optionally, the access catheter 1100 may draw the pericardium away from the heart HW to create or increase the pericardial space. At step 1240, the longitudinal member 1150 is positioned in the access catheter 1100 such that an energy delivery portion 1151 is positioned at the targeted portion TR. The longitudinal member 1150 comprises an insulated portion 1152, and a connector that is configured to transfer energy from an energy source (not shown) to the energy delivery portion 1151. At step 1250, energy is then communicated to the targeted portion TR to ablate or weaken the targeted portion TR. At step 1260, and as seen in FIG. 13C, an access opening PCO is created on the pericardium such that the pericardial space and the heart HW may be accessed.

It is further envisioned that the two or more longitudinal members may be used to deliver ablation energy to the targeted portion as seen in FIG. 14. As seen in FIG. 14, the targeted portion TR of the pericardium PCM is isolated by the suction force communicated from the suction port 2370. Thereafter, two longitudinal members 2350 and 2360 are positioned in the access catheter 2300 such that the two energy delivery portions 2351 and 2361 are substantially in contact with the targeted portion TR. In this arrangement, the energy delivery portion 2351 first longitudinal member 2350 may act as the cathode and the energy delivery portion 2361 of the second longitudinal member 2360 may act as anode such that controlled energy deployment may be achieved using a bipolar arrangement of the two energy delivery portions. Thereafter, energy is delivered to the targeted portion TR such that an access opening may be created as previously described.

In the various embodiments described above, the access catheter may be configured to be compatible with a variety of longitudinal members, for example longitudinal members having a diameter of 0.010 inches, 0.014 inches, 0.018 inches, 0.035 inches, 0.038 inches, or longitudinal members having other diameters. Alternatively, the access catheter may be configured to be compatible with a smaller or larger diameter longitudinal member. Optionally, the catheter or catheter system as described herein may be used in combination with other devices such as various endoscopic devices, angioplasty devices, etc. Additionally and optionally, the catheter or catheter system may comprise additional lumens, and/or lumens of sufficient diameter or size, to accommodate various elements such as visualization elements, therapeutic agent delivery elements, etc., to increase treatment effectiveness.

Furthermore, in the various embodiments described above, the access catheter may comprise radiopaque markers disposed on or near the proximal end and/or the distal end. The radiopaque markers may facilitate tracking the position of the catheter, particularly, while the catheter navigates through narrow and/or tortuous vasculature during the operation. Additionally, the longitudinal member may comprise radiopaque markers disposed on or near the proximal end and/or the distal end to enable tracking as well.

While the above is a complete description of the preferred embodiments of the invention, various alternatives, modifications, and equivalents may be used. Therefore, the above description should not be taken as limiting the scope of the invention which is defined by the appended claims.

What is claimed is:

1. A method for accessing the heart, comprising:
advancing an access catheter through up to a portion of the pericardium;
stabilizing a distal end of the access catheter on the portion of the pericardium; advancing a longitudinal member from the access catheter, wherein the longitudinal member comprises at least one energy delivery portion;
delivering energy through the energy delivery portion of the longitudinal member to the portion of the pericardium, wherein an opening is created in the portion of the pericardium; and accessing the heart through the opening in the portion of the pericardium.

2. The method of claim 1, wherein the stabilizing further comprises applying suction through the access catheter to the portion of the pericardium.

3. The method of claim 1, further comprising separating the portion of the pericardium from the heart.

4. The method of claim 1, wherein the energy delivery portion of the longitudinal member is configured to delivery radio-frequency energy to the portion of the pericardium.

5. The method of claim 1, further comprising sealing the opening in the portion of the pericardium by cauterizing the opening using energy delivered through the energy delivery portion of the longitudinal member
